(19) United States
(12) Patent Application Publication
(10) Pub. No.: US 2005/0064401 A1
(43) Pub. Date: Mar. 24, 2005

(54) Diagnosis of illnesses or predisposition to certain illnesses

(76) Inventors: Alexander Olek, Berlin (DE); Christian Piepenbrock, Berlin (DE); Kurt Berlin, Stahnsdorf (DE)

Correspondence Address: KRIEGSMAN & KRIEGSMAN, 665 FRANKLIN STREET, FRAMINGHAM, MA, 01702, US

(21) Appl. No.: 10/363,483

(22) PCT Filed: Sep. 1, 2001
(86) PCT No.: PCT/EP01/10073

(30) Foreign Application Priority Data
Sep. 1, 2000 (DE) 100 43 826.1

(51) Int. Cl. C12Q 1/68; C07H 21/04; C07K 16/00

(52) U.S. Cl. 435/6; 530/324; 536/24.3

(57) ABSTRACT

The present invention describes a set of oligomer probes (oligonucleotides and/or PNA oligomers), which serve for the detection of the cytosine methylation state in nucleic acids. These probes are particularly suitable for the diagnosis of existing diseases by analysis of a set of genetic and/or epigenetic parameters.

### FIELD OF THE INVENTION

The levels of observation that have been well studied in molecular biology according to developments in methods in recent years include the genes themselves, the transcription of these genes into RNA and the translation to proteins therefrom. During the course of development of an individual, which gene is turned on and how the activation and inhibition of certain genes in certain cells and tissues are controlled can be correlated with the extent and nature of the methylation of the genes or of the genome. In this regard, pathogenic states are also expressed by a modified methylation pattern of individual genes or of the genome.

The present invention describes nucleic acids, oligonucleotides, PNA oligomers and a method for the diagnosis of existing diseases or of predisposition for specific diseases.

### PRIOR ART

The methylation of CpG islands is often equated with transcription inactivity. Although there is clear evidence that CpG islands are to be found in promoters of genes, not all CpG islands and methylation sites are localized in known promoters. In different tissue-specific and imprinting genes, the CpG islands are localized at considerable distances downstream of the start of transcription, and also many genes possess multiple promoters. For a number of diseases, methylation of CpG dinucleotides has been detected as a causal factor. In contrast to classical mutations, DNA methylation involves a mechanism that describes a base substitution without modifying the coding function of a gene. This interplay between epigenetic modification and classical mutations plays an important role in tumorigenesis. For example, focal hypermethylation and generalized genomic demethylation are features of many different tumor types. It is assumed that tumorigenesis and tumor progression are caused, first of all, by hypermethylation of induced mutation events, and secondly, by the turning off of genes which control cellular proliferation and/or by the induced reactivation of genes, which are [normally] used only for embryological development, via demethylation.

In hereditable non-polyposis colorectal cancer, e.g., the majority of mutation-negative cases of colon cancer are based rather on the hypermethylation of the hMLH1 promoter and the associated non-expression of hMLH1, a repair gene for erroneous base pairings (Bevilacqua R A, Simpson A J, Methylation of the hMLH1 promoter but no hMLH1 mutations in sporadic gastric carcinomas with high-level microsatellite instability. Int J Cancer. Jul. 15, 2000 ;87(2):200-3.). In the pathogenesis of lung cancer, the loss of expression is correlated with the methylation of CpG islands in the promoter sequence of an RAS effector homolog. (Dammann R, Li C, Yoon J H, Chin P L, Bates S, Pfeifer G P, Nucleotide. Epigenetic inactivation of a RAS association domain family protein from the lung tumour suppressor locus 3p21.3. Nat. Genet. July 2000 ;25(3):315-9). An epigenetic inactivation of the LKB1 tumor supressor gene, including the hypermethylation of the promoter, is associated with the Peutz-Jeghers syndrome (Esteller M, Avizienyte E, Corn P G, Lothe R A, Baylin S B, Aaltonen L A, Herman J G, Epigenetic inactivation of LKB1 in primary tumors associated with the Peutz-Jeghers syndrome. Oncogene. Jan. 6, 2000;19(1):164-8).

A plurality of diseases, which are associated with methylation, have in their etiology a close connection with the tumor suppressor genes p16 or p15. Thus a relationship between Mycosis fungoides and hypermethylation of the p16(INK4a) gene is assumed (Navas I C, Ortiz-Romero P L, Villuendas R, Martinez P, Garcia C, Gomez E, Rodriguez J L, Garcia D, Vanaclocha F, Iglesias L, Piris M A, Algara P, p16(INK4a) gene alterations are frequent in lesions of mycosis fungoides. Am J Pathol. May, 2000; 156(5):1565-72). Also, there is a strong correlation between the turning off of the transcription of the p16 gene in gastric carcinoma and the de novo methylation of a few specific CpG sites (Song S H, Jong H S, Choi H H, Kang S H, Ryu M H, Kim N K, Kim W H, Bang Y J, Methylation of specific CpG sites in the promoter region could significantly down-regulate p16(INK4a) expression in gastric adenocarcinoma. Int J Cancer. Jul. 15, 2000;87(2):236-40). The pathogenesis of cholangiocarcinoma, which is associated with primary sclerosing cholangitis, has been related to the inactivation of the p16 tumor suppressor gene, which is again dependent on the methylation of the p16 promoter (Ahrendt S A, Eisenberger C F, Yip L, Rashid A, Chow J T, Pitt H A, Sidransky D, Chromosome 9p21 loss and p16 inactivation in primary sclerosing cholangitis-associated cholangiocarcinoma. J Surg Res. Jun. 1, 2000;84(1):88-93). The inactivation of the p16 gene by hypermethylation plays a role in the genesis of leukemia and in the progression of acute lymphoblastic leukemia (Nakamura M, Sugita K, Inukai T, Goi K, Iijima K, Tezuka T, Kojika S, Shiraishi K, Miyamoto N, Karakida N, Kagami K, O-Koyama T, Mori T, Nakazawa S, p16/MTS1/INK4A gene is frequently inactivated by hypermethylation in childhood acute lymphoblastic leukemia with 11q23 translocation. Leukemia. June 2000;13(6):884-90). In addition, it is postulated that the hypermethylation of the p16 and p15 genes plays a decisive role in the tumorigenesis of multiple myeloma (Ng M H, Wong I H, Lo K W, DNA methylation changes and multiple myeloma. Leuk Lymphoma. August 1999;34(5-6):463-72). The VHL gene, which is inactivated by methylation, appears to participate in predisposition to renal carcinoma (Glavac D, Ravnik-Glavac M, Ovcak Z, Masera A, Genetic changes in the origin and development of renal cell carcinoma (RCC). Pflugers Arch. 1996;431(6 Suppl 2):R193-4). A divergent methylation of the 5′-CpG island may participate in nasopharyngeal carcinoma, possibly by the inactivation of transcription of the p16 gene (Lo K W, Cheung S T, Leung S F, van Hasselt A, Tsang Y S, Mak K F, Chung Y F, Woo J K, Lee J C, Huang D P, Hypermethylation of the p16 gene in nasopharyngeal carcinoma. Cancer Res. Jun. 15, 1996;56(12):2721-5). An inactivation of the p16 protein was detected in liver cell carcinoma. Promoter hypermethylation and homozygous deletions are the most frequent mechanisms here (Jin M, Piao Z, Kim N G, Park C, Shin E C, Park J H, Jung H J, Kim C G, Kim H, p16 is a major inactivation target in hepatocellular carcinoma. Cancer. Jul. 1, 2000;89(1):60-8). DNA methylation as a control of gene expression was detected for the BRCA1 gene for breast cancer (Magdinier F, Billard L M, Wittmann G, Frappart L, Benchaib M, Lenoir G M, Guerin J F, Dante, R Regional methylation of the 5′ end CpG island of BRCA1 is associated with reduced gene expression in human somatic cells FASEB J. August 2000;14(11):1585-94). A correlation between methylation and non-Hodgkin's lymphoma is also assumed (Martinez-Delgado B, Richart A, Garcia M J, Robledo M, Osorio A, Cebrian A, Rivas C, Benitez J, Hypermethylation of P16ink4a and P15ink4b genes as a marker of disease in the follow-up of non-Hodgkin's lymphomas. Br J Haematol. April 2000;109(1):97-103).

CpG methylation also brings about the progression of T-cell leukemia, which is related to a decreased expression of the CDKN2A gene (Nosaka K, Maeda M, Tamiya S, Sakai T, Mitsuya H, Matsuoka M, Increasing methylation of the CDKN2A gene is associated with the progression of adult T-cell leukemia. Cancer Res. Feb. 15, 2000;60(4):1043-8). An increased methylation of the CpG islands was established in bladder cancer (Salem C, Liang G, Tsai Y C, Coulter J, Knowles M A, Feng A C, Groshen S, Nichols P W, Jones P A, Progressive increases in de novo methylation of CpG islands in bladder cancer. Cancer Res. May 1, 2000;60(9):2473-6). Transcription inactivation in esophageal squamous cell carcinomas has been related to the methylation of the FHIT gene, which is associated with the progression of the disease (Shimada Y, Sato F, Watanabe G, Yamasaki S, Kato M, Maeda M, Imamura M, Loss of fragile histidine triad gene expression is associated with progression of esophageal squamous cell carcinoma, but not with the patient's prognosis and smoking history. Cancer. Jul. 1, 2000;89(1):5-11). Neutral endopeptidase 24.11 (NEP) inactivates the increase of neuropeptides, which participate in the growth of androgen-independent prostate cancer. A loss of NEP expression by hypermethylation of the NEP promotors may contribute to the development of neuropeptide-stimulated, androgen-independent prostate cancer (Usmani B A, Shen R, Janeczko M, Papandreou C N, Lee W H, Nelson W G, Nelson J B, Nanus D M, Methylation of the neutral endopeptidase gene promoter in human prostate cancers. Clin Cancer Res. May 2000;6(5):1664-70). Adrenocortical tumors in adults display structural abnormalities in the tumor DNA. Among other things, these abnormalities contain an overexpression of the IGF2 gene in correlation with a demethylation of the DNA at this locus (Wilkin F, Gagne N, Paquette J, Oligny L L, Deal C, Pediatric adrenocortical tumors: molecular events leading to insulin-like growth factor 11 gene overexpression. J Clin Endocrinol Metab. May 2000;85(5):2048-56. Review). It is assumed that DNA methylations in several exons in the retinoblastoma gene contribute to the disease (Mancini D, Singh S, Ainsworth P, Rodenhiser D, Constitutively methylated CpG dinucleotides as mutation hot spots in the retinoblastoma gene (RB1). Am J Hum Genet. July 2000;61(1):80-7). In chronic myeloid leukemia, a relationship is suspected between the deregulation of the p53 gene and a change in the methylation pattern with progression of the disease (Guinn B A, Mills K I, p53 mutations, methylation and genomic instability in the progression of chronic myeloid leukaemia. Leuk Lymphoma. July 1997;26(3-4):211-26). A relationship with methylation has also been detected for acute myeloid leukemia (Melki J R, Vincent P C, Clark S J. Concurrent DNA hypermethytation of multiple genes in acute myeloid leukemia. Cancer Res. Aug. 1, 1999;59(15):3730-40). A tumor-specific methylation site in the Wilms tumor suppressor gene has been identified (Kleymenova E V, Yuan X, LaBate M E, Walker C L, Identification of a tumor-specific methylation site in the Wilms tumor suppressor gene. Oncogene. Feb. 12, 1998;16(6):713-20). In Burkitt's lymphoma, several promotors have a complete CpG methylation (Tao Q, Robertson K D, Manns A, Hildesheim A, Ambinder R F, Epstein-Barr virus (EBV) in endemic Burkitt's lymphoma: molecular analysis of primary tumor tissue. Blood. Feb. 15, 1998;91(4):1373-81). It is assumed that DNA methylation plays a role in thyroid carcinoma (Venkataraman G M, Yatin M, Marcinek R, Ain K B, Restoration of iodide uptake in dedifferentiated thyroid carcinoma: relationship to human Na+/I-symporter gene methylation status. J Clin Endocrinol Metab. July 1999;84(7):2449 57).

Not only are many cancer diseases associated with methylation, but there are also many other diseases that are related to methylation. Investigations of inflammatory arthritis have indicated that this disease is associated with a hypomethylation of genomic DNA (Kim Y I, Logan J W, Mason J B, Roubenoff R, DNA hypomethylation in inflammatory arthritis: reversal with methotrexate. J Lab Clin Med. August 1996;128(2):165-72). A methylation-regulated expression has been detected for the ICF syndrome (Kondo T, Bobek M P, Kuick R, Lamb B, Zhu X, Narayan A, Bourc'his D, Viegas-Pequignot E, Ehrlich M, Hanash S M, Whole-genome methylation scan in ICF syndrome: hypomethylation of nonsatellite DNA repeats D4Z4 and NBL2). The participation of methylation is suspected in systemic lupus erythematosus (Vallin H, Perers A, Alm G V, Ronnblom L, Anti-double-stranded DNA antibodies and immunostimulatory plasmid DNA in combination mimic the endogenous IFN-alpha inducer in systemic lupus erythematosus. J Immunol. December 1999;163(11):6306-13); and there may also be a relationship between the Duchenne muscular dystrophy gene and a CpG-rich island (Banerjee S, Singh P B, Rasberry C, Cattanach B M, Embryonic inheritance of the chromatin organisation of the imprinted H19 domain in mouse spermatozoa. Mech Dev. February 2000;90(2):217-26; Burmeister M, Lehrach H, Long-range restriction map around the Duchenne muscular dystrophy gene. Nature. Dec. 11-17, 1986;324(6097):582-5). An epigenetic effect, in which the hypomethylation of the amyloid precursor protein [gene], which is related to the development of the disease, participates, is suspected in Alzheimer's disease (West R L, Lee J M, Maroun L E, Hypomethylation of the amyloid precursor protein gene in the brain of an Alzheimer's disease patient. 1995;6(2):141-6). The methylation state also plays an important role at the chromosomal level. For example, in mental retardation syndromes that are associated with the fragility of the X chromosome, the degree of chromosomal fragility is determined by the methylation (de Muniain A L, Cobo A M, Poza J J, Saenz A, [Diseases due to instability of DNA]. Neurologia. December 1995;10 Suppl 1:12-9).

5-Methylcytosine is the most frequent covalently modified base in the DNA of eukaryotic cells. For example, it plays a role in the regulation of transcription, in genetic imprinting and in tumorigenesis. The identification of 5-methylcytosine as a component of genetic information is thus of considerable interest. 5-Methylcytosine positions, however, cannot be identified by sequencing, since 5-methylcytosine has the same base-pairing behavior as cytosine. In addition, in the case of a PCR amplification, the epigenetic information which is borne by the 5-methylcytosines is completely lost.

A relatively new method that in the meantime has become the most widely used method for investigating DNA for 5-methylcytosine is based on the specific reaction of bisulfite with cytosine, which, after subsequent alkaline hydrolysis, is then converted to uracil, which corresponds in its base-pairing behavior to thymidine. In contrast, 5-methylcytosine is not modified under these conditions. Thus, the original DNA is converted so that methylcytosine, which originally cannot be distinguished from cytosine by its hybridization behavior, can now be detected by “standard” molecular biology techniques as the only remaining cytosine, for example, by amplification and hybridization or sequencing. All of these techniques are based on base pairing, which is now fully utilized. The prior art, which concerns sensitivity, is defined by a method that incorporates the DNA to be investigated in an agarose matrix, so that the diffusion and renaturation of the DNA is prevented (bisulfite reacts only on single-stranded DNA) and all precipitation and purification steps are replaced by rapid dialysis (Olek, A. et al., Nucl. Acids Res. 1996, 24, 5064-5066). Individual cells can be investigated by this method, which illustrates the potential of the method. Of course, up until now, only individual regions of up to approximately 3000 base pairs long have been investigated; a global investigation of cells for thousands of possible methylation analyses is not possible. Of course, this method also cannot reliably analyze very small fragments of small quantities of sample. These are lost despite the protection from diffusion through the matrix.

An overview of other known possibilities for detecting 5-methylcytosines can be derived from the following review article: Rein, T., DePamphilis, M. L., Zorbas, H., Nucleic Acids Res. 1998, 26, 2255.

The bisulfite technique has been previously applied only in research, with a few exceptions (e.g., Zechnigk, M. et al., Eur. J. Hum. Gen. 1997, 5, 94-98). However, short, specific segments of a known gene have always been amplified after a bisulfite treatment and either completely sequenced (Olek, A. and Walter, J., Nat. Genet 1997, 17, 275-276) or individual cytosine positions have been detected by a primer extension reaction (Gonzalgo, M. L. and Jones, P. A., Nucl. Acids Res. 1997, 25, 2529-2531, WO-Patent 95-00669) or an enzyme step (Xiong, Z. and Laird, P. W., Nucl. Acids Res. 1997, 25, 2532-2534). Detection by hybridization has also been described (Olek et al., WO-A 99-28,498).

Other publications which are concerned with the application of the bisulfite technique for the detection of methylation in the case of individual genes are: Xiong, Z. and Laird, P. W. (1 7)*, Nucl. Acids Res. 25, 2532; (Gonzalgo, M. L. and Jones, P. A., (1997), Nucl. Acids Res. 25, 2529; Grigg, S. and Clark, S. (1994), Bioassays 16, 431; Zeschnik, M. et al. (1997), Human Molecular Genetics 6, 387; Teil, R. et al. (1994), Nucl. Acids Res. 22, 695; Martin, V. et al. (1995), Gene 157, 261; WO-A 97-46,705, WO-95-15,373 and WO-45,560.
(1987)?—Trans. Note.

Matrix-assisted laser desorptions/ionization mass spectrometry (MALDI-TOF) is a very powerful development for the analysis of biomolecules (Karas, M. und Hillenkamp, F. (1988). Laser desorption Ionization of proteins with molecular masses exeeding 10000 daltons. Anal. Chem. 60: 2299-2301). An analyte is embedded in a light-absorbing matrix. The matrix is vaporized by a short laser pulse and the analyte molecule is transported unfragmented into the gaseous phase. The analyte is ionized by collisions with matrix molecules. An applied voltage accelerates the ions in a field-free flight tube. Ions are accelerated to varying degrees based on their different masses. Smaller ions reach the detector sooner than large ions.

MALDI-TOF spectroscopy is excellently suitable for the analysis of peptides and proteins. The analysis of nucleic acids is somewhat more difficult (Gut, I. G. and Beck, S. (1995)), DNA and Matrix Assisted Laser Desorption Ionization Mass Spectrometry. Molecular Biology: Current Innovations and Future Trends 1: 147-157). For nucleic acids, the sensitivity is approximately 100 times poorer than for peptides and decreases overproportionally with increasing fragment size. For nucleic acids, which have a backbone with a multiple negative charge, the ionization process via the matrix is basically less efficient. In MALDI-TOF spectroscopy, the choice of matrix plays an imminently important role. Several very powerful matrices, which produce a very fine crystallization, have been found for the desorption of peptides. In the meantime, several effective matrices have been developed for DNA, but the difference in sensitivity has not been reduced thereby. The difference in sensitivity can be reduced by modifying the DNA chemically in such a way that it resembles a peptide. Phosphorothioate nucleic acids, in which the usual phosphates of the backbone are substituted by thiophosphates, can be converted by simple alkylation chemistry to a charge-neutral DNA (Gut, I. G. and Beck, S. (1995), A procedure for selective DNA alkylation and detection by mass spectrometry. Nucleic Acids Res. 23: 1367-1373). The coupling of a charge tag to this modified DNA results in an increase in sensitivity to the same order of magnitude as is found for peptides. Another advantage of charge tagging is the increased stability of the analysis in the presence of impurities, which make the detection of unmodified substrates very difficult.

Genomic DNA is obtained from DNA of cells, tissue or other test samples by standard methods. This standard methodology is found in references such as Fritsch and Maniatis, eds., Molecular Cloning: A Laboratory Manual, 1989.

### Presentation of the Problem

The present invention will present oligonucleotides and/or PNA oligomers for the detection of cytosine methylation and a method, which is particularly suitable for the diagnosis of existing diseases or of predisposition for specific diseases by analysis of a set of genetic and/or epigenetic parameters.

### DESCRIPTION

The present invention describes a set of at least 10 oligomer probes (oligonucleotides and/or PNA oligomers), which serve for the detection of the cytosine methylation state in chemically pretreated genomic DNA (Seq. ID 1 to Seq. ID 40712). The analysis of a set of genetic and/or epigenetic parameters for the diagnosis of existing diseases or for the diagnosis of predisposition to specific diseases is possible with these probes.

Genetic parameters in the sense of this invention are mutations and polymorphisms of the claimed nucleic acids (Seq. ID 1 to Seq. ID 40712) and additional sequences necessary for their regulation. Particularly designated as mutations are insertions, deletions, point mutations, inversions and polymorphisms and particularly preferred are SNPs (single nucleotide polymorphisms). Polymorphisms, however, can also be insertions, deletions or inversions.

Epigenetic parameters in the sense of this invention are particularly cytosine methylations and other chemical modifications of DNA bases of the claimed nucleic acids (Seq. ID 1 to Seq. ID 40712) and additional sequences necessary for their regulation. Other epigenetic parameters, for example, are the acetylation of histones, although this cannot be directly analyzed with the described method; however, it is correlated in turn with DNA methylation. From said chemically pretreated DNA, segments that are at least 18 base pairs in length from Seq. ID 1 to Seq. ID 40712 are utilized for the diagnosis. Oligomers with a length of at least 9 nucleotides are used as detectors of these segments.

The oligomers contain at least one CpG dinucleotide. The cytosine of the corresponding CpG dinucleotide is found in approximately the middle third of the oligomer. It is a deciding factor that at least one oligonucleotide from Seq. ID 1 to Seq. ID 40712 is present in the respective set of oligomers for at least each of the CpG dinucleotides. The oligomers are preferably produced on a support material in a fixed arrangement, whereby at least one oligomer is coupled to a solid phase.

It is also important in this connection that it is not individual CpG dinucleotides, but the plurality of CpG dinucleotides present in the sequences, which must be analyzed for the diagnosis of genetic and/or epigenetic parameters of the claimed nucleic acids (Seq. ID 1 to Seq. ID 40712). In a particularly preferred variant of the method, all of the CpG dinucleotides present in the sequences are to be investigated.

In a preferred variant of the method, at least one oligomer is bound to a solid phase.

In another preferred variant of the method, at least ten of the oligomers are used for the detection of the cytosine methylation state and/or of single nucleotide polymorphisms (SNPs) in chemically pretreated genomic DNA.

The oligomers are preferably used for the diagnosis of undesired drug interactions; cancer diseases; CNS malfunctions, damage or disorders; symptoms of aggression or behavioral disturbances; clinical, psychological and social consequences of brain lesions; psychotic disturbances and personality disorders; dementia and/or associated syndromes; cardiovascular disease; malfunction, damage or disorder of the gastrointestinal tract; malfunction, damage or disease of the respiratory system; lesion, inflammation, infection, immunity and/or convalescence; malfunction, damage or disease of the body as an abnormality in the development process; malfunction, damage or disorder of the skin, the muscles, the connective tissue or the bones; endocrine and metabolic malfunction, damage or disease; headaches and sexual malfunctions, by analysis of methylation patterns.

Also, of the nucleic acids listed in the Appendix (Seq. ID 1 to Seq. ID 40712), preferably at least one will be used for the analysis of a set of genetic and/or epigenetic parameters for the diagnosis of existing diseases or for the diagnosis of predisposition for specific diseases.

In addition, a method is described for determining important genetic and/or epigenetic parameters for the diagnosis of existing diseases or for the diagnosis of predisposition for specific diseases, by analysis of cytosine methylations and of single nucleotide polymorphisms (SNPs) in chemically pretreated genomic DNA samples. The procedure for this comprises the following steps:

In the first step of the method, a genomic DNA sample is chemically treated in such a way that cytosine bases that are unmethylated at the 5′-position are converted to uracil, thymine or another base unlike cytosine in its hybridization behavior. This is understood in the following as chemical pretreatment.

The person of average skill in the art understands that the oligomers fulfill the same objective when thymine is exchanged for uracil in the sequences used.

The genomic DNA to be analyzed is obtained preferably from the usual sources for DNA, such as, e.g., cell lines, blood, sputum, stool, urine, cerebrospinal fluid, tissue embedded in paraffin, for example, tissue from eyes, intestine, kidney, brain, heart, prostate, lung, breast or liver, histological slides and all other possible combinations thereof.

Preferably, the above-described treatment of genomic DNA with bisulfite (hydrogen sulfite, disulfite) and subsequent alkaline hydrolysis, which converts unmethylated cytosine nuleobases to uracil, is used for this purpose.

In the second step of the method, fragments from the chemically pretreated genomic DNA are amplified with the use of primer oligonucleotides.

Preferably, more than 10 different fragments are amplified, which are 100-2000 base pairs in length.

In a preferred variant of the method, the amplification is preferably conducted with the polymerase chain reaction (PCR), wherein a heat-stable DNA polymerase is preferably used.

It is preferred according to the invention that the amplification of several DNA segments is conducted in one reaction vessel.

In a preferred variant of the method, the set of primer oligonucleotides comprises at least two oligonucleotides, whose sequences are inversely complementary or identical to a segment that is at least 18 base pairs long of the base sequences listed in the Appendix (Seq. ID 1 to Seq. ID 40712). The primer oligonucleotides are preferably characterized in that they do not contain a CpG dinucleotide.

According to the invention, it is also preferred that different oligomers are arranged on a planar solid phase in the form of a rectangular or hexagonal grid.

In a preferred variant of the method, the amplification occurs by elongation of primer oligonucleotides that are bound to a solid phase.

This solid-phase surface is preferably comprised of silicon, glass, polystyrene, aluminum, steel, iron, copper, nickel, silver, or gold.

The amplified products obtained in the second step are then hybridized to a set of oligonucleotides and/or PNA probes or to an array. The set used in the hybridization is most preferably comprised of at least 10 oligomer probes. The amplified products thus serve as probes, which hybridize to the oligonucleotides previously bound to a solid phase. The unhybridized fragments are then removed.

Said oligomers comprise at least one base sequence with a length of 9 nucleotides, which contains at least one CpG dinucleotide. The cytosine of the corresponding CpG dinucleotide is found in approximately the middle third of the oligomer. One oligonucleotide is present for each CpG dinucleotide.

In the fourth step of the method, the unhybridized amplified products are removed.

In the last step of the method, the hybridized amplified products are detected.

It is preferred according to the invention that labels, which are introduced on the amplified products at any position of the solid phase at which an oligonucleotide sequence is found, can be identified.

It is preferred according to the invention that the labels of the amplified products are fluorescent labels.

It is preferred according to the invention that the labels of the amplified products are radionuclides.

It is preferred according to the invention that the labels of the amplified products are removable molecular fragments with typical mass, which are detected in a mass spectrometer.

It is preferred according to the invention that the amplified products, fragments of the amplified products or probes complementary to the amplified products are detected in the mass spectrometer.

It is preferred according to the invention that the produced fragments have a single positive or negative net charge for better detectability in the mass spectrometer.

It is preferred according to the invention that the detection is carried out and visualized by means of matrix-assisted laser desorption/ionization mass spectrometry (MALDI) or by means of electrospray mass spectrometry (ESI).

A method is preferred for the diagnosis and/or prognosis of adverse events for patients or individuals, whereby these adverse events are related to genetic and/or epigenetic parameters.

The use of a method according to the invention is preferred for the diagnosis of existing diseases or of the predisposition for specific diseases by analysis of a set of genetic and/or epigenetic parameters.

The subject of the present invention is also a kit comprising a reagent containing bisulfite, a set of primer oligonucleotides comprising at least two oligonucleotides, each of whose sequences is a segment that is at least 18 base pairs long and corresponds to the base sequences listed in the Appendix (Seq. ID 1 to Seq. ID 40712) or are complementary to them for the production of amplified products, oligonucleotides and/or PNA oligomers as well as instructions for conducting and evaluating the described method.

The following example relates to a fragment of the hMLH1 gene associated with hereditable non-polyposis colorectal cancer, in which a specific CG position is investigated for methylation.

In the first step, a genomic sequence is treated with the use of bisulfite (hydrogen sulfite, disulfite) in such a way that all of the unmethylated cytosines at the 5-position of the base are modified such that a base that is different in its base pairing behavior is formed, while the cytosines that are methylated in the 5-position remain unchanged. If bisulfite in the concentration range between 0.1 M and 6 M is used for the reaction, then an addition occurs at the unmethylated cytosine bases. Also a denaturing reagent or solvent as well as a radical trap must be present. A subsequent alkaline hydrolysis then leads to the conversion of unmethylated cytosine nucleobases to uracil. This converted DNA serves for the detection of methylated cytosines. In the second step of the method, the treated DNA sample is diluted with water or an aqueous solution. A desulfonation of the DNA (10-30 min, 90-100° C.) at alkaline pH is then preferably conducted. In the third step of the method, the DNA sample is amplified in a polymerase chain reaction, preferably with a heat-stable DNA polymerase. In the present example, cytosines of the hMLH1 gene, here from a 1551 bp-long 5′-flanking region, are investigated. A defined fragment of 719-bp length is amplified for this purpose with the specific primer oligonucleotides AGCMCACCTCCATGCACTG and TTGATTGGACAGCTTGAATGC. This amplified product serves as a sample, which hybridizes to an oligonucleotide that has been previously bound to a solid phase, with the formation of a duplex structure, for example, GAAGAGCGGACAG, whereby the cytosine to be detected is found at position 588 of the amplified product. The detection of the hybridization product is based on primer oligonucleotides fluorescently labeled with Cy3 and Cy5, which were used for the amplification. A hybridization reaction of the amplified DNA with the oligonucleotide occurs only if a methylated cytosine was present at this site in the bisulfite-treated DNA. Thus the methylation state of the respective cytosine to be investigated decides the hybridization product.

1. Nucleic acids comprising a sequence segment at least 18 bases long of a chemically pretreated DNA according to one of the sequences Seq. ID 1 to Seq. ID 40712.

2. An oligomer (oligonucleotide or peptide nucleic acid (PNA) oligomer) for the detection of the cytosine methylation state in chemically pretreated DNA, containing at least one base sequence with a length of at least 9 nucleotides, which hybridizes to a chemically pretreated DNA (Seq. ID 1 to Seq. ID 40712).

3. The oligomer according to claim 2, whereby the base sequence comprises at least one CpG dinucleotide.

4. The oligomer according to claim 3, further characterized in that the cytosine of the CpG dinucleotide is found in approximately the middle third of the oligomer.

5. A set of oligomers according to claim 3, comprising at least one oligomer for at least one of the CpG dinucleotides of one of the sequences of Seq. ID 1 to Seq. ID 40712.

6. A set of oligomers according to claim 5 containing at least one oligomer for each of the CpG dinucleotides of one of the sequences of Seq. ID 1 to Seq. ID 40712.

7. A set of at least two nucleic acids according to claim 2, which are utilized as primer oligonucleotides for the amplification of DNA sequences according to at least one of the sequences Seq. ID 1 to Seq. ID 40712 or segments thereof.

8. A set of oligonucleotides according to claim 7, further characterized in that at least one oligonucleotide is bound to a solid phase.

9. A set of oligomer probes for the detection of the cytosine methylation state and/or of single nucleotide polymorphisms (SNPs) in chemically pretreated genomic DNA according to one of the sequences Seq. ID 1 to Seq. ID 40712, comprising at least ten of the oligomers according to one of claims 2 to 4.

10. A method for the production of an arrangement of different oligomers (an array) fixed on a support material for the analysis of disorders related to the methylation state of the CpG dinucleotides of one of the sequences Seq. ID 1 to Seq. ID 40712, in which at least one oligomer according to one of claims 2 to 4 is coupled to a solid phase.

11. An arrangement of different oligomers (an array) according to one of claims 2 to 4, which is bound to a solid phase.

12. The array of different oligonucleotide and/or PNA oligomer sequences according to claim 11, further characterized in that these are arranged on a planar solid phase in the form of a rectangular or hexagonal grid.

13. The array according to claim 11, further characterized in that the solid phase surface is comprised of silicon, glass, polystyrene, aluminum, steel, iron, copper, nickel, silver, or gold.

14. A DNA and/or PNA array for the analysis of disorders related to the methylation state of genes, which contains at least one nucleic acid according to one of claims 1 or 2.

15. A method for determining genetic and/or epigenetic parameters for the diagnosis of existing diseases or of the predisposition for specific diseases by analysis of cytosine methylations, is hereby characterized in that the following steps are conducted:
a) in a genomic DNA sample, cytosine bases that are unmethylated at the 5′-position are converted by chemical treatment to uracil or another base unlike cytosine in its base-pairing behavior;
b) from this chemically pretreated genomic DNA, fragments are amplified with the use of sets of primer oligonucleotides according to claim 7 or 8 and a polymerase, whereby the amplified products bear a detectable label;
c) the amplified products are hybridized to a set of oligonucleotides and/or PNA probes containing at least one base sequence with a length of at least 9 nucleotides which hybridizes to a chemically pretreated DNA (Seq. ID 1 to Seq. ID 40712) or, however, to an array of different such oligonucleotides and/or PNA probes bound to a solid phase;
d) the hybridized amplified products are then detected.

16. The method according to claim 15, further characterized in that the chemical treatment is conducted by means of a solution of a bisulfite, hydrogen sulfite or disulfite.

17. The method according to claim 15, further characterized in that more than ten different fragments are amplified, which are 100-2000 base pairs in length.

18. The method according to claim 15, further characterized in that the amplification of several DNA segments is conducted in one reaction vessel.

19. The method according to claim 15, further characterized in that the polymerase is a heat-stable DNA polymerase.

20. The method according to claim 18, further characterized in that the amplification is conducted by means of the polymerase chain reaction (PCR).

21. The method according to claim 15, further characterized in that the labels of the amplified products are fluorescent labels.

22. The method according to claim 15, further characterized in that the labels of the amplified products are radionuclides.

23. The method according to claim 15, further characterized in that the labels of the amplified products are removable molecular fragments with typical mass, which are detected in a mass spectrometer.

24. The method according to claim 15, further characterized in that the amplified products or fragments of the amplified products are detected in the mass spectrometer.

25. The method according to claim 23, further characterized in that the produced fragments have a single positive or negative net charge for better detectability in the mass spectrometer.

26. The method according to 23, further characterized in that the detection is carried out and visualized by means of matrix-assisted laser desorption/ionization mass spectrometry (MALDI) or by means of electrospray mass spectrometry (ESI).

27. The method according to claim 15, further characterized in that the genomic DNA was obtained from cells or cell components that contain DNA, whereby sources for DNA comprise e.g., cell lines, biopsies, blood, sputum, stool, urine, cerebrospinal fluid, tissue embedded in paraffin, for example, tissue from eyes, intestine, kidney, brain, heart, prostate, lung, breast or liver, histological slides and all possible combinations thereof.

28. A kit, comprising a bisulfite (=bisulfite (disulfite), hydrogen sulfite) reagent as well as oligonucleotides and/or PNA oligomers according to one of claims 2 to 4.

29. Use of a nucleic acid comprising a sequence segment at least 18 bases long of a chemically pretreated DNA according to one of the sequences Seq. ID 1 to Seq. ID 40712, an oligonucleotide or PNA oligomer containing at least one base sequence with a length of at least 9 nucleotides which hybridizes to a chemically pretreated DNA (Seq. ID 1 to Seq. ID 40712), a kit comprising a bisulfite (=bisulfite (disulfite), hydrogen sulfite) reagent as well as oligonucleotides and/or PNA oligomers according to one of claims 2 to 4, or an array of such oligonucleotides and/or PNA oligomers fixed on a support material for the diagnosis and/or therapy of undesired drug interactions; cancer diseases; CNS malfunctions; symptoms of aggression or behavioral disturbances; clinical, psychological and social consequences of brain lesions; psychotic disturbances and personality disorders; dementia and/or associated syndromes; cardiovascular disease; malfunction, damage or disorder of the gastrointestinal tract; malfunction, damage or disorder of the respiratory system; lesion, inflammation, infection, immunity and/or convalescence; malfunction, damage or disease of the body as an abnormality in the development process; malfunction, damage or disorder of the skin, the muscles, the connective tissue or the bones; endocrine and metabolic malfunctions, headaches; sexual malfunctions, by analysis of methylation patterns.

30. The method according to claim 24, further characterized in that the produced fragments have a single positive or negative net charge for better detectability in the mass spectrometer.

31. The method according to claim 24, further characterized in that the detection is carried out and visualized by means of matrix-assisted laser desorption/ionization mass spectrometry (MALDI) or by means of electrospray mass spectrometry (ESI).
